(19) United States
(12) Patent Application Publication
(10) Pub. No.: US 2017/0014304 A1
(43) Pub. Date: Jan. 19, 2017

(54) FINGER-PRESSURE TOOL FOR FACE

(71) Applicant: SEILIN & CO., Tokyo (JP)

(72) Inventor: Yoshiko TARUMI, Tokyo (JP)

(21) Appl. No.: 15/278,104

(22) Filed: Sep. 28, 2016

Related U.S. Application Data
(63) Continuation-in-part of application No. 13/881,046, filed on Apr. 23, 2013.

(30) Foreign Application Priority Data
Oct. 25, 2010 (JP) 2010-238437
Nov. 29, 2010 (JP) PCT/JP2010/071209

(51) Int. Cl. A61H 39/04; A61F 9/02; A61H 1/00

(52) U.S. Cl. CPC A61H 39/04; A61H 1/006; A61F 9/026; A61F 9/029; A61H 2201/0111; A61H 2201/0115; A61H 2201/1604; A61H 2201/165; A61H 2205/022; A61H 2205/024

(57) ABSTRACT

Provided is a facial finger-pressure tool capable of accelerating alleviation of asthenopia. A finger-pressure protrusion (8) is provided on an inner side of a front portion (11) and a temple portion (17) of a goggles frame (1), at a position corresponding to at least one acupuncture point around an eye, wherein the goggles frame (1) is made of a flexible material.

### CROSS-REFERENCE TO RELATED APPLICATION

This application is a continuation-in-part of U.S. patent application Ser. No. 13/881,046 filed on Apr. 23, 2013, which was based upon and claimed the benefit of priority from the prior PCT/JP2010/071209 filed on Nov. 29, 2010; and Japanese Patent Application No. JP 2010-238437 filed on Oct. 25, 2010, the entire contents of which are incorporated herein by reference.

### TECHNICAL FIELD

The present invention relates to a facial finger-pressure tool, and more specifically to a facial finger-pressure tool which is capable of accurately applying pressing stimulation to a facial pressure point, and is effective, particularly, in reducing asthenopia.

### BACKGROUND ART

It is commonly and widely known that a prolonged period of detailed-oriented manual work, personal computer work, automobile driving or TV viewing causes asthenopia and then headache, shoulder stiffness, etc.

Meanwhile there are several acupuncture points (pressure points) around a human eye, and stimulation of the acupuncture point (pressure point) improves particularly blood circulation around the eye, thereby making it possible to accelerate alleviation of asthenopia.

With a focus on this point, JP 08-304743A discloses an invention concerning an eyeglass designed to magnetize an inner surface of an eyeglass frame, i.e., a surface of an eyeglass frame facing a face of a user of the eyeglass.

### LIST OF PRIOR ART DOCUMENTS

### Patent Documents

Patent Document 1: JP 08-304743A

### SUMMARY OF THE INVENTION

### Technical Problem

In the device disclosed in the above Patent Document, a magnetic source is disposed on an inner surface of a conventional eyeglass frame. However, as a way to improve blood circulation around an eye, it is also effective to apply finger-pressure to an acupuncture point (pressure point) around the eye, as well as the technique of applying a magnetic field thereto. A magnetic therapy needs to be continued for a certain period of time, because it does not have an immediate effect. The magnetic means is desirable as prophylactic use. However, in view of accelerating alleviation of asthenopia, it is most effective to directly stimulate an acupuncture point (pressure point) around an eye.

The conventional device is not provided with any member for stimulating the acupuncture point (pressure point), i.e., adapted to be brought into direct and press contact with a face. Thus, there is a problem that such a finger-pressure effect cannot be expected.

Moreover, the conventional device is intended to offer improvement based on an eyeglass frame, so that it is an uninteresting article to non-eyeglass wearers.

### Solution to the Technical Problem

The present invention has been made with a focus on the above problem of the conventional technique, and proposed with a view to providing a facial finger-pressure tool capable of accelerating the alleviation of asthenopia.

In order to achieve the above object, the present invention provides a facial finger-pressure tool which comprises: a goggles frame; and a finger-pressure protrusion provided on an inner surface of a front portion and a temple portion of the goggles frame, at a position corresponding to at least one acupuncture point around an eye, wherein the goggles frame is made of a flexible material.

The present invention also provides a facial finger-pressure tool which comprises: a goggles frame; and a finger-pressure protrusion provided on an inner surface of a front portion and a temple portion of the goggles frame, at a position corresponding to at least one acupuncture point around an eye, wherein the goggles frame is made of a flexible material, and the temple portion has two free ends provided with tightening means adapted to forcibly bring the goggles frame into close contact with face and head regions.

The present invention also provides a facial finger-pressure tool which consists of a front frame adapted to cover an eye region and provided with a finger-pressure protrusion on an inner surface thereof, at a position corresponding to at least one acupuncture point around an eye, wherein the front frame is made of a flexible material.

The present invention also provides a facial finger-pressure tool which consists of an eye mask-like body adapted to cover an eye region, and provided with a finger-pressure protrusion on an inner surface thereof, at a position corresponding to an acupuncture point around an eye.

In each of the above facial finger-pressure tools of the present invention, the finger-pressure protrusion is attached adjustably and movably in conformity to a position of the acupuncture point.

In each of the above facial finger-pressure tools of the present invention, the finger-pressure protrusion is detachably attached.

In each of the above facial finger-pressure tools of the present invention, the goggles frame is made of a flexible material which is at least one selected from the group consisting of polyvinyl chloride, soft synthetic resin, natural rubber, and synthetic rubber including chloroprene rubber, silicon rubber, acrylic rubber and urethane rubber.

In each of the above facial finger-pressure tools of the present invention, the finger-pressure protrusion is comprised of a metal tablet or a magnet.

### Effect of the Invention

In the facial finger-pressure tool in accordance with the present invention, the finger-pressure protrusion is provided on the inner surface of the front portion and the temple portion of the goggles frame, at a position corresponding to at least one acupuncture point around an eye, and the goggles frame is made of a flexible material, so that the finger-pressure protrusion can be pressed against the acupuncture point to apply adequate stimulation to the acupuncture point, which is significantly effective in curing asthenopia. In addition, when a user feels that the stimulation is insufficient in a normal usage state, he/she can press the goggles frame from an outside thereof toward the face by the palm. This provides stronger stimulation and an enhanced finger-pressure effect.

In the facial finger-pressure tool, the tightening means can be provided at the two free ends of the temple portion, so that a level of tightness can be adjusted to adjust a level of press contact (force) of the finger-pressure protrusion against the facial acupuncture point. That is, when the tightening means is tightly fastened (tied), the goggles frame made of a flexible material is more strongly brought into close contact with face and head regions to increase the press contact force of the finger-pressure protrusion against the facial acupuncture point, thereby providing a further enhanced stimulation (finger-pressure) effect. Further, the adjustment of the tightness makes it possible to obtain any level of finger-pressure effect according to user's preference.

In accordance with one aspect of the present invention, the facial finger-pressure tool can include a front frame which is adapted to cover an eye region and provided with a finger-pressure protrusion on an inner surface of the front portion, at a position corresponding to at least one acupuncture point around an eye, wherein the front frame is made of a flexible material.

Thus, for example, a cord or a tensile member such as a rubber band may be attached to the front frame to allow the front frame to be brought into press contact with the face. Alternatively, the front frame may be used in a state in which it is interposed between the face and existing swimming goggles, 3D TV glasses or the like. Further, it may be used in various other manners. For example, a user may place only this facial finger-pressure tool on the eye region, and then move the palm in a direction for pressing the front portion and in a direction away from the front portion.

In accordance with another feature of the present invention, the facial finger-pressure tool can be formed as an eye mask-like, i.e., wide-width body. Thus, a large number of finger-pressure protrusions may be provided thereon. In this case, the finger-pressure protrusions can be brought into contact with points other than acupuncture points, so that beauty and health effects of facial massage can be expected in addition to stimulation of acupuncture points.

In the facial finger-pressure tool, the finger-pressure protrusion can be attached in a positionally changeable manner. Thus, the position of the finger-pressure protrusion can be finely adjusted to adequately conform to a position of the acupuncture point. This makes it possible to obtain a further adequate finger-pressure effect.

In the facial finger-pressure tool as set forth in the appended claim 6, the finger-pressure protrusion is detachably attached. Thus, a size, such as contact area and/or height dimension, of the finger-pressure protrusion can be freely changed to obtain an adequate finger-pressure effect.

In the facial finger-pressure tool, the front portion and the temple portion of the goggles frame can be made of a flexible material which is at least one selected from the group consisting of polyvinyl chloride, soft synthetic resin, natural rubber, and synthetic rubber including chloroprene rubber, silicon rubber, acrylic rubber and urethane rubber, so that the goggles frame can be brought into close contact with the face to obtain an adequate finger-pressure effect.

In the facial finger-pressure tool, the finger-pressure protrusion can be comprised of a magnet, or a NOIROKORUN brand metal tablet which is a trade name of Ryodoraku Research Institute, Ltd., so that it becomes possible to apply stimulation to an acupuncture point in a close contact state with the face, thereby obtaining an adequate finger-pressure effect. This metal tablet is a metal tablet-shaped body, and used in physical therapy as an applied technique of principles of acupuncture and finger-pressure.

### BRIEF DESCRIPTION OF THE DRAWINGS

FIG. 1 is a perspective view illustrating a first embodiment of the present invention.

FIG. 2 is an explanatory diagram illustrating an example of attachment of a finger-pressure protrusion.

FIG. 3 is a perspective view illustrating a second embodiment of the present invention.

FIG. 4 is a perspective view illustrating a third embodiment of the present invention.

FIG. 5 is a diagram illustrating acupuncture points around an eye.

FIG. 6 is a perspective view illustrating the grooves of the present invention.

### DESCRIPTION OF EMBODIMENTS

An embodiment of the present invention will now be described based on the drawings.

### First Embodiment

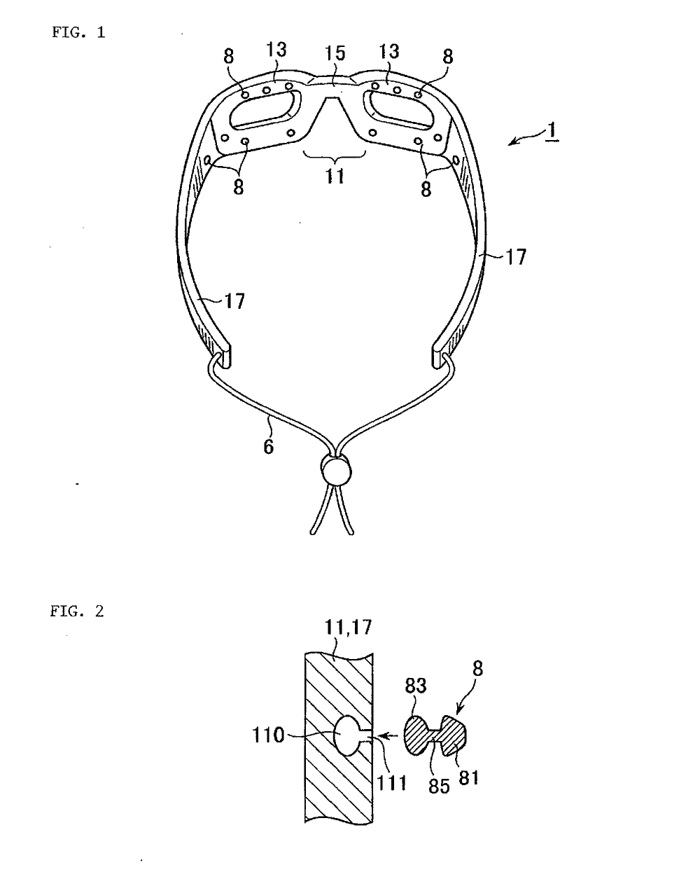

FIG. 1 illustrates a first embodiment of the present invention, wherein a goggles frame 1 as a face finger-pressure tool comprises a front portion 11 and a pair of temple portions 17. The front portion 11 is comprised of a pair of rims 13 and a bridge 15 connecting the rims 13 together, as called in the field of eyeglass frames. In this embodiment, no nose pad is provided. Thus, an inner (face-facing) surface of the front portion 11 is brought into close contact with a face, as described later.

The temple portions 17 are integrally connected, respectively, to opposite ends of the front portion 11. The front portion 11 and the pair of the temple portions 17 may be integrally molded in a single piece, or may be molded separately and subsequently connected together. In the case where they are molded separately, it is desirable to connect them together by means of hinge connection or the like.

A pair of tightening members 6 such as rubber belts or straps are attached, respectively, to ear-side ends, i.e., free ends, of the temple portions 17. When the right and left tightening members 6 are integrally fastened (locked) on a rear side of a head, the goggles frame is brought into close contact with face and temporal regions. As the tightening members are tightly fastened (tied), a level of press contact of an aftermentioned finger-pressure protrusion 8 becomes higher, and a level of stimulation becomes stronger in proportion thereto. Thus, a user can adjustably increase and reduce a level of the tightness according to his/her preference.

The front portion 11 and the temple portions 17 of the goggles frame are made of a flexible material which is at least one selected from the group consisting of: soft synthetic resin such as polyurethane, polyvinyl chloride or acrylonitrile; and natural or synthetic rubber such as silicon rubber or chloroprene rubber.

A protrusion 8 for a finger-pressure effect (finger-pressure protrusion 8) is attached to the inner surface of the front portion 11 and the temple portions 17. The attachment position of the finger-pressure protrusion 8 corresponds to: an acupuncture point (pressure point), such as “seimei” (a depressed region located slightly away from inner corners of right and left eyes toward a nose), “gyoyou” (a position just below a center of an eyebrow), “taiyou” (a position close to a tail of the eye with respect to a temple), or “shihaku” (a position just below a center of a pupil); or an acupuncture point (pressure point) around the eye, such as “shichikuku”, “doushiryou”, “joumei”, “gaimei”, “kyugo”, “shoukyu” or “sanchiku” (see FIG. 2). Asthenopia, and headache, shoulder stiffness, etc., to be caused by the asthenopia, can be alleviated by applying stimulation to such an acupuncture point.

FIG. 2 illustrates one example of attachment of the finger-pressure protrusion 8, wherein the finger-pressure protrusion 8 comprises a pressing portion 81, a fitting engagement portion 83, and a bridge portion 85 connecting them together. A hole 110 for holding the finger-pressure protrusion 8 is formed in the inner surface of the front portion 11 and the temple portion 17, at a position corresponding to the acupuncture point. The hole 110 is configured to have a size capable of fastenably receiving therein the fitting engagement portion 83 of the finger-pressure protrusion 8.

An inlet portion 111 of the hole 110 is formed to have a reduced diameter, thereby fastenably holding the bridge portion 85 of the finger-pressure protrusion 8. This prevents the finger-pressure protrusion 8 from being easily unfastened or disengaged from the inner surface of the front portion 11 and the temple portion 17. Alternatively, the finger-pressure protrusion 8 may be adhesively attached, for example, using an adhesive seal.

A plurality of types of the finger-pressure protrusions 8 each different in size, such as contact area and/or height dimension, of the pressing portion 81 to be brought into contact with the facial acupuncture point, may be prepared in advance. In this case, a level of finger-pressure can be freely adjusted according to user's preference.

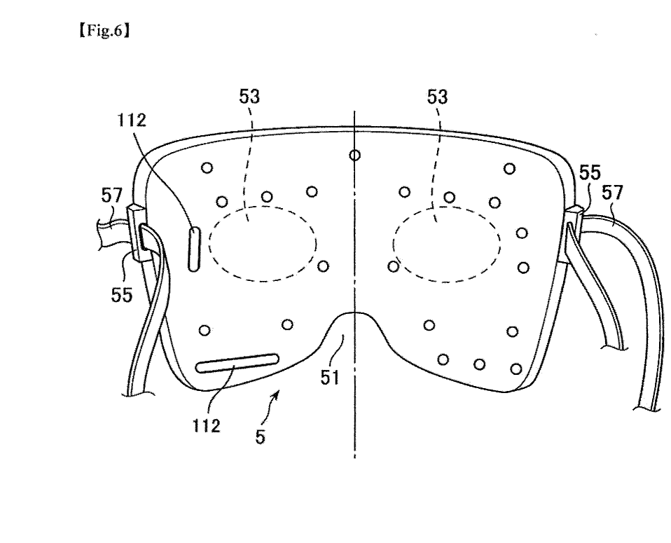

Further, the hole 110 (111) formed in the front portion 11 and the temple portions 17 may be formed in a shape extending in a right-left or front-rear direction and/or an up-down direction, e.g., a cross shape as shown in FIG. 6. That is, at least one groove 112 may be also formed in the front portion and/or the temple portions. The at least one groove 112 may extend in a horizontal direction and/or a vertical direction. Similar to the hole 110 (111), the groove 112 has an inlet portion reduced in the width direction, wherein only the inlet portion is used so as to fastenably receive therein a fitting engagement portion of the finger-pressure protrusion. In this case, the finger-pressure protrusion 8 can be moved or slid in the horizontal direction and/or vertical direction along the groove in a continuous manner, which makes it possible to adequately conform to a position of the acupuncture point in an easy manner.

The finger-pressure protrusion 8 should be provided in a number equal to that of the acupuncture points as a minimum requirement. However, the present invention is not limited thereto, but the finger-pressure protrusion 8 may be provided at a position other than the positions of the acupuncture points. In this case, beauty and health effects as effects of massaging the entire eye region can be expected.

The finger-pressure protrusion 8 desirably made of, but is not particularly limited to, a metal material, preferably, a NOIROKORUN brand metal tablet which is a trade name of Ryodoraku Research Institute, Ltd. which is a magnetized metal material. That is, in cases where it is desired to obtain only a finger-pressure effect without any need for magnetic therapy, a substitute material such as a hard plastic may be used.

The above tightening members 6 are not provided in the facial finger-pressure tool 1 in claim 1. In this case, a user may press the front portion 11 and/or the temple portions 17 by his/her palms, to obtain a finger-pressure effect. As an example of the tightening member, it is possible to use a rubber band, a strap or a cord.

While any object, such as a lens, is not installed inside each of the rims 13 of the front portion 11 of the facial finger-pressure tool, a lens for visual correction, a lens for 3D glasses or the like may be installed. In this case, it is necessary to additionally take measure to provide conventional lens fixing/holding means to the rims.

### Second Embodiment

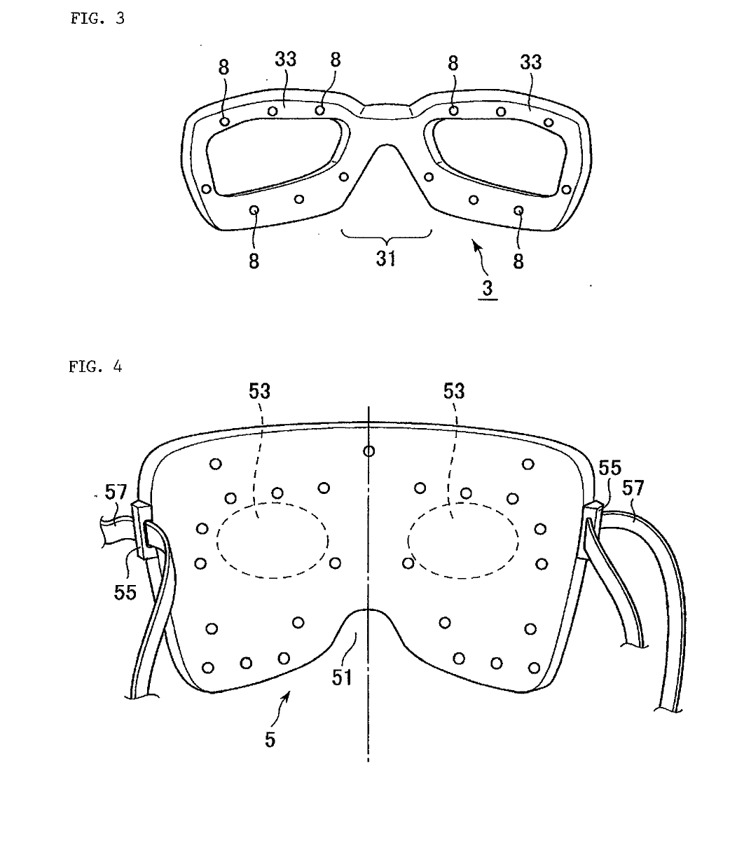

FIG. 3 illustrates a second embodiment wherein a facial finger-pressure tool 3 consists only of a front frame 31. The front frame 31 is comprised of a pair of rims 33 and a bridge 35 integrally connecting the rims 33 together. That is, the facial finger-pressure tool is similar to horn-rimmed glasses in terms of configuration.

As with the first embodiment, a finger-pressure protrusion 8 is installed to an inner surface of the front frame 31. As with the first embodiment, the finger-pressure protrusion 8 may be fastenably attached, or may be detachably attached, or may be configured to be displaceable along the inner surface in conformity to a position of the acupuncture point. The front frame 31 may have a hole provided at each of opposite ends thereof to allow a rubber belt, a strap or the like to be attached thereto.

In the facial finger-pressure tool illustrated in FIG. 3, the front frame 31 can be fixed to a face by a rubber belt, a strap or the like. In the case where such fixing means is not used, the front frame 31 may be interposed between a face and 3D TV glasses, swimming goggles or the like, in such a manner as to be brought into press contact with the face by means of a holding force of the 3D TV glasses, the swimming goggles or the like, to obtain a finger-pressure effect. For this purpose, the front frame 31 is in a plain state in which no lens is installed.

In the case of using swimming goggles, it is necessary to take measures to allow the facial finger-pressure tool 3 to be reliably received in the swimming goggles, while preventing intrusion of water through the swimming goggles. Such water-tightness can be ensured by forming them using substantially the same material.

### Third Embodiment

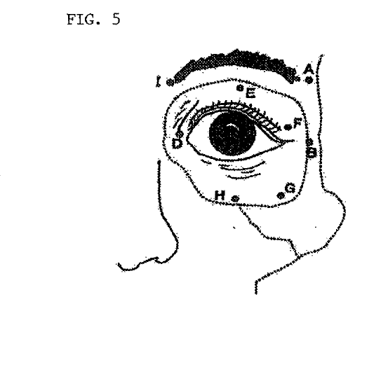

FIG. 4 illustrates a facial finger-pressure tool according to a third embodiment, wherein the facial finger-pressure tool 5 is formed as an eye mask-like body adapted to cover a part of a cheek region as well as an eye region. In the illustrated embodiment, the reference numeral 51 indicates a region for allowing a nose to be exposed. The reference numeral 53 indicates a transparent portion for allowing an eye to be exposed. Alternatively, the transparent portion 53 may be omitted as in a sleeping eye mask.

The reference numeral 55 indicates each of a pair of strap mounts, and the reference numeral 57 indicates each of a pair of straps to be fastened together on a rear side of a head so as to maintain the facial finger-pressure tool 5 in a contact state with the face. In the case where the facial finger-pressure tool 5 is not held by the head, the straps 57 may be omitted, and the facial finger-pressure tool 5 may be used for massage in such a manner that it is placed on a face and pressed by a palm. It is to be understood that, even in the case where the facial finger-pressure tool 5 is kept in close contact with the face by the straps, it may be pressed by a palm when it is desired to apply a stronger pressing force.

A finger-pressure protrusion 8 is provided on an inner surface of the facial finger-pressure tool 5. The finger-pressure protrusion 8 is provided at a position corresponding to that of the acupuncture point as with the above embodiments. Differently from the facial finger-pressure tool illustrated in FIG. 1 or 3, the facial finger-pressure tool 5 has a wider contact area with a face. Thus, the finger-pressure protrusion 8 is appropriately provided at a position other than that of the acupuncture point. That is, a finger-pressure protrusion at a position corresponding to the acupuncture point is used for stimulation of a pressure point, and a finger-pressure protrusion at other position is used for a facial massage.

As with the above embodiments, the facial finger-pressure tool 5 is made of at least one selected from the group consisting of polyvinyl chloride, soft synthetic resin, natural rubber, and synthetic rubber such as chloroprene rubber, silicon rubber, acrylic rubber and urethane rubber. As means for attachment of the finger-pressure protrusion 8, the configuration as illustrated in FIG. 2, or adhesive attachment, may be employed. Further, the finger-pressure protrusion 8 may be comprised of a magnet.

This embodiment is not limited to the eye mask-like body, but may be a Noh mask-like body adapted to cover the entire face. In this case, a massage effect for the entire face can be obtained, which is advantageous to beauty and health. The massage may be performed in such a manner that the facial finger-pressure tool is pressed by a palm, or lightly touched by a vibrator type electric massaging device.

### EXPLANATION OF CODES

1: facial finger-pressure tool
11: front portion
17: temple portion
3: facial finger-pressure tool
31: front frame
33: rim
35: bridge
5: facial finger-pressure tool
53: transparent portion
55: strap mount
57: strap
6: tightening member
8: finger-pressure protrusion
81: pressing portion
83: fitting engagement portion
85: bridge portion
A: shichikuku
B: doushiryou
D: seimei
E: joumei
F: gaimei
G: kyugo
H: shoukyu
I: sanchiku

What is claimed is:

1. A facial finger-pressure tool comprising: a goggles frame; and a finger-pressure protrusion provided on an inner surface of a front portion and a temple portion of the goggles frame, at a position corresponding to at least one acupuncture point around an eye, wherein the goggles frame is made of a flexible material, and the temple portion has two free ends provided with tightening means adapted to forcibly bring the goggles frame into close contact with face and head regions, the facial finger-pressure tool being characterized in that the goggles frame has holes which are formed in the inner surface of the front and temple portions at the position corresponding to the acupuncture points or at other positions, each of the holes having an inlet portion reduced in diameter so as to fastenably receive therein a fitting engagement portion of the finger-pressure protrusion, the finger-pressure protrusion being attached movably in conformity to a position of any one of the holes, at least one groove which is formed in the front portion and/or the temple portions, said groove extending in a horizontal direction and/or vertical direction so that the finger-pressure protrusion is movable in the horizontal direction and/or the vertical direction along the groove in a continuous manner so as to conform the finger pressure protrusion adequately to a position of the acupuncture point in an easy manner, said groove having an inlet portion reduced in width direction, wherein only said inlet portion is used so as to fastenably receive therein a fitting engagement portion of the finger-pressure protrusion, and the facial finger-pressure tool is provided with a plurality of types of the finger-pressure protrusions each different in at least contact area size and height dimension of the pressing portion to be brought into contact with the facial acupuncture point and the various types of the finger-pressure protrusions are replaceable for any one of the holes so that a level of finger-pressure can be freely adjusted according to a user's preference.

2. The facial finger-pressure tool as defined in claim 1, wherein the finger-pressure protrusion is detachably attached.

3. The facial finger-pressure tool as defined in claim 1, wherein a shape of the goggles frame is formed in a shape of an eye mask adapted to cover an eye region.

4. The facial finger-pressure tool as defined in claim 1, wherein the finger-pressure protrusion is made of a material which is one selected from the group consisting of metal, hard plastic, magnetized metal, and a metal tablet.
